(19) United States
(12) Patent Application Publication
(10) Pub. No.: US 2023/0112140 A1
(43) Pub. Date: Apr. 13, 2023

(54) ELECTROCHEMICAL SENSOR, CONTINUOUS ANALYTE METER INCLUDING ELECTROCHEMICAL SENSOR, AND METHOD OF FABRICATING ELECTROCHEMICAL SENSOR

(71) Applicant: UXN Co., Ltd., Gwangju (KR)

(72) Inventor: Jun Young PARK, Gyeonggi-do (KR)

(21) Appl. No.: 17/961,657

(22) Filed: Oct. 7, 2022

(30) Foreign Application Priority Data
Oct. 7, 2021 (KR) 10-2021-0132955
Sep. 13, 2022 (KR) 10-2022-0114856

(51) Int. Cl. A61B 5/1473; A61B 5/00

(52) U.S. Cl. CPC A61B 5/1473; A61B 5/6849; A61B 2562/125

(57) ABSTRACT

An electrochemical sensor includes a distal portion on which a plurality of electrodes configured to react with an in vivo analyte are provided, a proximal portion on which sensor pads connected to the electrodes are provided, and an intermediate portion positioned between the distal portion and the proximal portion. A transmitter includes a main substrate on which at least one of a power source, a communication unit, and a controller is provided and a housing in which the main substrate is accommodated. The transmitter is configured to be attached to the skin. A method of fabricating an electrochemical sensor includes applying a conductive layer on a flexible base layer of the electrochemical sensor and attaching insulating layers to the conductive layer.

### CROSS REFERENCE TO RELATED APPLICATION AND CLAIM OF PRIORITY

This application claims the benefit under 35 USC § 119 of Korean Patent Application Nos. 10-2021-0132955, filed on Oct. 7, 2021, and 10-2022-0114856 filed on Sep. 13, 2022, in the Korean Intellectual Property Office, the entire disclosure of which is incorporated herein by reference for all purposes.

### BACKGROUND

### 1. Field of the Invention

The present disclosure relates generally to an electrochemical sensor, at least a portion of which is inserted into a body (e.g., a human body), a continuous analyte meter including the electrochemical sensor, and a method of fabricating the electrochemical sensor.

### 2. Description of the Related Art

When the position of an inserter is set as a reference position, one end of an electrochemical sensor connected to a main substrate may be referred to as a proximal portion since the position thereof is close to the inserter. The other end of the electrochemical sensor to be inserted into a body (e.g., a human body) may be referred to as a distal portion since the position thereof is remote from the inserter.

The proximal portion of the electrochemical sensor may be electrically connected to a main substrate of a transmitter, and at least a portion of the distal portion of the electrochemical sensor may be inserted into a body. The proximal portion and the distal portion may be positioned on opposite ends. The proximal portion of the electrochemical sensor may be electrically connected to the main substrate of the transmitter including an electrical circuit necessary for measuring an analyte including glucose.

The base layer of the electrochemical sensor may be flexible in order to reduce pain caused by an invasion and reduce foreign body feeling when attached to the body. It is necessary to minimize the thickness and size of the electrochemical sensor.

The smaller the size of the electrochemical sensor, the smaller the area of an electrode provided on the distal portion may be. When the area of the electrode is not sufficient, signal disturbance may be caused by noise. Thus, in fabrication of an electrochemical sensor, both aspects of the size reduction of the sensor and a sufficient electrode area of the sensor need to be considered.

The size of the electrochemical sensor is required to be minimized in order to reduce pain caused by an invasion and reduce foreign body feeling. The smaller the size of the electrochemical sensor, the smaller the area of the electrode provided on the distal portion may be. Since the area of the electrode is not sufficiently obtained, signal disturbance may be caused by noise. Thus, in fabrication of an electrochemical sensor, the trade-off relationship between the size reduction of the sensor and the sufficient electrode area is required to be satisfied.

The foregoing is intended merely to aid in the understanding of the background of the present disclosure, and is not intended to mean that the present disclosure falls within the purview of the related art that is already known to those skilled in the art.

### SUMMARY

Accordingly, the present disclosure has been made keeping in mind the above problems occurring in the related art, and the present disclosure is intended to propose an electrochemical sensor and a method of fabricating the electrochemical sensor, wherein a sufficient area may be obtained for electrodes supposed to react with an in vivo analyte while the size of the electrochemical sensor is reduced.

The electrochemical sensor included in a continuous analyte meter according to the present disclosure may have via-holes extending through a base layer of the electrochemical sensor. The via-holes according to the present disclosure may electrically connect sensor pads of the proximal portion and the electrodes of the distal portion provided on both surfaces of the electrochemical sensor.

In order to achieve at least one of the above objectives, a continuous analyte meter according to the present disclosure may include: an electrochemical sensor including a distal portion on which a plurality of electrodes configured to react with an in vivo analyte are provided, a proximal portion on which sensor pads connected to the electrodes are provided, and an intermediate portion positioned between the distal portion and the proximal portion; and a transmitter including a main substrate on which at least one of a power source, a communication unit, and a controller is provided and a housing in which the main substrate is accommodated, the transmitter being configured to be attached to the skin.

The distal portion of the electrochemical sensor may be provided on a portion exposed in a longitudinal direction of a needle. After the skin is cut by the needle, the distal portion of the electrochemical sensor may be inserted into a body. The electrochemical sensor may include a flexible base layer, a conductive layer applied on the base layer, and insulating layers attached on top of the conductive layer.

A method of fabricating an electrochemical sensor according to the present disclosure may include: applying a conductive layer on a flexible base layer of the electrochemical sensor; and attaching insulating layers to the conductive layer.

An electrochemical sensor according to the present disclosure may include: a substrate including a proximal portion on which a plurality of electrodes and leads extending from the electrodes are provided, with a plurality of sensor pads connected to the leads being provided on a top surface of the substrate, and a distal portion configured to be inserted into a body. The plurality of electrodes may include at least one top electrode provided on a top surface of the distal portion of the substrate and at least one bottom electrode provided on a bottom surface of the distal portion of the substrate. The leads include top leads and bottom leads may extend from the top electrodes and the bottom electrodes to coplanar portions of the proximal portion. Conductive structures may include some of the sensor pads of the proximal portion extending through the substrate to be electrically connected to the bottom leads.

In addition, an electrochemical sensor according to the present disclosure may include: a substrate including a proximal portion on which a plurality of sensor pads are provided and a distal portion to be inserted into a body; at least one top electrode provided on a top surface of the distal portion of the substrate and at least one bottom electrode provided on a bottom surface; top leads extending from the top electrodes, respectively, to the sensor pads on the proximal portion; and a conductive structure extending through the distal portion of the substrate to electrically connect the at least one top electrode and the at least one bottom electrode.

According to the present disclosure, since the electrodes are disposed on both surfaces of the distal portion, the electrodes or the leads may have a sufficient area. Accordingly, it is possible to reduce the defective rate caused by short-circuits, improve the sensitivity of the electrochemical sensor, reduce matching defects when forming the conductive layer and the insulating layers on the base layer, and reduce the length and width of insertion of the invasive distal portion.

According to the present disclosure, since the electrodes are disposed on both surfaces of the distal portion, it is possible to obtain a wide electrode area while reducing the width of the distal portion, reduce pain and foreign body feeling caused by the invasion, and increase electrochemical reactivity between the electrodes and an in vivo analyte due to the wide electrode area.

The electrochemical sensor according to the present disclosure may increase electrical reliability of a biosensor while providing an electrical connecting terminal portion with a simple structure.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a top perspective view illustrating a cross-section of an embodiment in which an inserter, an electrochemical sensor, and a transmitter are coupled according to the present disclosure;

FIG. 2 is an exploded perspective view illustrating a base layer, a conductive layer, and insulating layers of the electrochemical sensor in a first aspect according to the present disclosure;

FIG. 3 is a perspective view illustrating trenches and conductive islands according to the first aspect of the present disclosure;

FIG. 4 is diagram illustrating a dummy portion or an inner trench between the conductive islands according to the first aspect of the present disclosure;

FIG. 5 is diagram illustrating a dummy portion or an inner trench between the leads according to the first aspect of the present disclosure;

FIG. 6 is a cross-sectional view illustrating the electrochemical sensor according to the first aspect of the present disclosure, with the electrodes and the sensor pads being provided on both surfaces of the electrochemical sensor;

FIG. 7 is a cross-sectional view illustrating an embodiment according to the first aspect of the present disclosure, in which via-holes are formed in the proximal portion;

FIG. 8 is plan view of FIG. 7;

FIG. 9 is plan view of FIG. 7;

(a) of FIG. 10 is a schematic view illustrating an embodiment according to the first aspect of the present disclosure, in which via-holes are formed in the proximal portion, and (b) of FIG. 10 is a schematic view illustrating an embodiment according to the first aspect of the present disclosure, in which via-holes are formed in the distal portion;

FIG. 11 is a process view sequentially illustrating a method of fabricating the electrochemical sensor according to the first aspect of the present disclosure;

FIG. 12 is a side cross-sectional view, a plan view, and a bottom view illustrating an embodiment according to a second aspect of the present disclosure;

FIG. 13 is a side cross-sectional view, a plan view, and a bottom view illustrating an embodiment according to the second aspect of the present disclosure; and

FIG. 14 is a table illustrating results obtained by measuring resistances of via-holes coated with a conductive material on the surface thereof according to the present disclosure.

### DETAILED DESCRIPTION

Hereinafter, a situation in which an electrochemical sensor 400 of the present disclosure is used in a continuous glucose monitoring system (CGMS) for measuring an interstitial fluid or a blood glucose concentration will be described as an embodiment. However, the CGMS of the present disclosure is not limited to the measurement of a glucose concentration in a body (e.g., a human body), but may be extended to a continuous analyte device that measures other biomarkers.

### Inserter and Transmitter

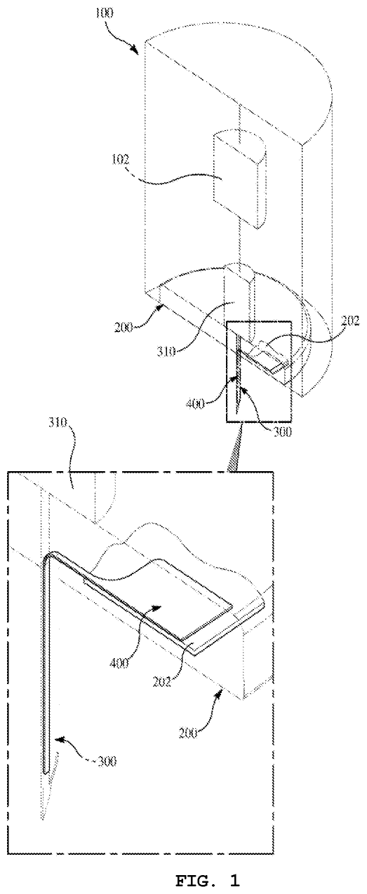

FIG. 1 illustrates one of combined embodiments between an inserter 100, an electrochemical sensor 400, and a transmitter 200 of the present disclosure. FIG. 1 may illustrate a state in which the electrochemical sensor 400 and the transmitter 200 are mounted inside the inserter 100 before being inserted into or being attached to the body after detached from the inserter 100.

Referring to FIG. 1, the electrochemical sensor 400 according to the present disclosure may be attached to the skin together with the transmitter 200. The transmitter 200 may control a signal measured by the electrochemical sensor 400 and transmit the continuously measured blood glucose level to an external terminal including a mobile device.

The external terminal may be provided separately from the transmitter 200 attached to the skin, and may continuously receive measurement data of the electrochemical sensor 400 wirelessly from the transmitter 200. A user may continuously monitor and diagnose the measurement data of the electrochemical sensor 400 for a bio-maker including glucose, lactate, and the like.

The electrochemical sensor 400 and the transmitter 200 may be provided to the user in a state of being loaded in the inserter 100 before being attached to the skin. By an attachment action by the user, the electrochemical sensor 400 and the transmitter 200 may be detached from the inserter 100 and attached to the skin.

One end of the electrochemical sensor 400 connected to electrical components 230 of the transmitter 200 may be referred to as a proximal portion 402, the other end of the electrochemical sensor 400, at least a portion of which is inserted into the body, is referred to as a distal portion 406, a portion disposed between the proximal portion 402 and the distal portion 406 to interconnect the proximal portion 402 and the distal portion 406 may be referred to as an intermediate portion 404, and a portion flexibly bent in the intermediate portion 404 so that the direction of the electrochemical sensor 400 is greatly changed may be referred to as a folded portion 405.

Invasion may mean insertion of at least a portion of the distal portion 406 of the electrochemical sensor 400 into the body.

The transmitter 200 and the electrochemical sensor 400 may be provided to the user in a pre-engaged state to each other before being attached to the skin.

The transmitter 200 is positioned in a first position in a state of being loaded in the inserter 100, and the transmitter 200 is moved by a user's action from the first position to a second position, at which the transmitter 200 is attached to the skin. The transmitter 200 and the electrochemical sensor 400 may be inserted in a direction from the first position to the second position.

A needle 300 has a portion exposed in the longitudinal direction, and a part of the electrochemical sensor 400 may be disposed inside the needle 300. The needle 300 may function to cut the skin and guide the electrochemical sensor 400 so that at least a portion of the distal portion 406 can be inserted into the human body along the insertion direction.

The inserter 100 may include a drive unit 102 that drives the transmitter 200 and the electrochemical sensor 400 from the first position to the second position.

The drive unit 102 may advance the needle 300 or transmitter 200 from the first position to the second position such that the needle 300 or the distal portion 406 is inserted into the skin.

After the transmitter 200 and the electrochemical sensor 400 are attached to the skin at the second position, the drive unit 102 retracts the needle 300 from the second position to a third position to separate the needle 300 from the transmitter 200 and the electrochemical sensor 400.

The drive unit 102 may be connected to a needle handle 310 to which the needle 300 is fixed. The needle handle 310 may be detachably attached to the drive unit 102.

An internal space may be provided between an upper housing 210 and a lower housing 220 of the transmitter 200.

One surface of the electrochemical sensor 400 on which sensor pads 428 are provided may face a main board 202, and the other surface of the electrochemical sensor 400 may be exposed to the internal space of the transmitter 200.

Contact pads 612 may be provided on the main board 202 so as to be electrically connected to the sensor pads 428 at the proximal portion 402 of the electrochemical sensor 400.

Since at least a portion of the electrochemical sensor 400 is inserted into the skin, the electrochemical sensor 400 or a base layer 410 may be flexible to relieve pain during invasion and reduce a feeling of foreign body when worn.

The distal portion 406 of the electrochemical sensor 400 may be disposed at a portion of the needle 300 exposed along the longitudinal direction thereof. An end of the needle 300 protrudes further from an end of the distal portion 406. After the skin is incised by the needle 300, the distal portion 406 of the electrochemical sensor 400 may be inserted into the body.

### Electrochemical Sensor

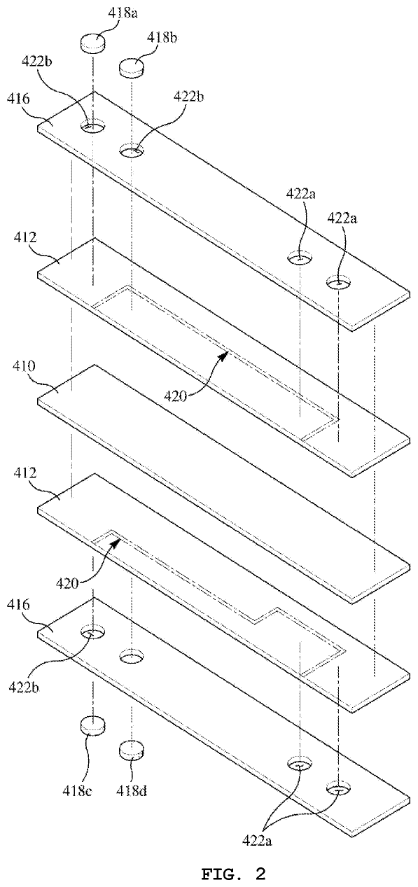

With reference to FIGS. 2 to 14, the electrochemical sensor 400 according to the present disclosure and a manufacturing method thereof will be described.

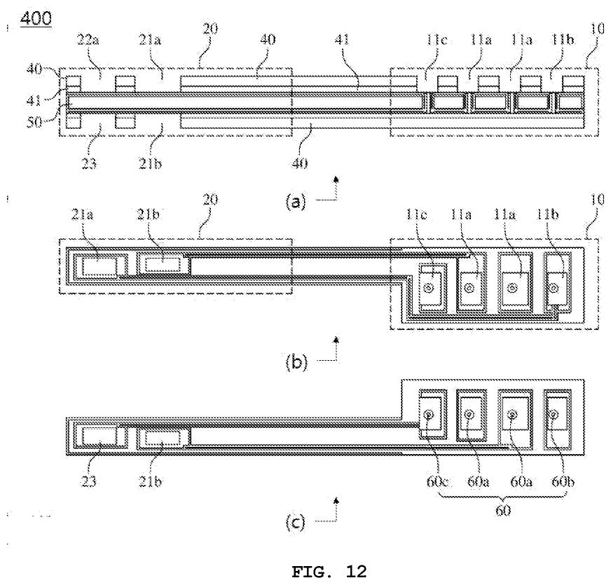

FIGS. 2 to 11 illustrate an electrochemical sensor 400 according to a first aspect, and FIGS. 12 to 14 illustrate an electrochemical sensor 400 according to a second aspect.

With reference to FIGS. 2 to 11, the first aspect will be described first.

According to the present disclosure, electrodes 424 and the sensor pads 428 may be provided on one side of the electrochemical sensor 400. In addition, the electrodes 424 and the sensor pads 428 may also be provided on both sides of the electrochemical sensor 400.

The electrochemical sensor 400 according to the present disclosure may selectively react with some of various analytes including glucose in the body through the electrodes 424 of the distal portion 406 to be inserted into the body.

When a voltage is applied to the electrodes 424 according to the present disclosure, an analyte in the body including glucose may be oxidized and reduced to generate electrons so that a current may flow. Since the generated current may be determined according to the concentration of an analyte in the body, a signal of a biomarker including a blood glucose level may be quantified.

The electrodes 424 may be provided on the distal portion 406, and the electrode may be inserted into the body so as to perform an oxidation or reduction reaction with sugar. The electrodes 424 may include at least one of a working electrode, a counter electrode, and a reference electrode.

The sensor pads 428 may be provided on the proximal portion 402 so as to be connected to the electrodes 424. An electric current generated through an electrochemical reaction with glucose in the body at the distal portion 406 may flow to the sensor pads 428 on the proximal portion 402 through leads 426 provided on the base layer 410. The sensor pads 428 may be in electrical communication with the main board 202 through the contact pad.

A plurality of leads 426 may be provided in the intermediate portion 404 so as to connect the electrodes 424 and the sensor pads 428. The plurality of leads 426 may be provided by laser etching to remove a portion of the conductive layer 412 by irradiating the conductive layer 412 with a laser beam. Accordingly, respective leads 426 may be arranged such that each lead does not cross or is twisted with another lead.

The electrodes 424 may include at least one working electrode and a reference electrode. The plurality of counter electrodes may be provided as needed. The counter electrode may be provided when three or more types of electrodes are used for precise data acquisition.

The working electrode may be a porous platinum (Pt) electrode and may be fabricated from a porous platinum colloid.

The reference electrode may be an electrode that has a constant potential and thus serves as a reference. The reference electrode may be one of a silver chloride (Ag/AgCl) electrode, a calomel electrode, and a mercury sulfate (I) electrode. When the biomarker is glucose, a silver chloride (Ag/AgCl) electrode may be used as the reference electrode for invasive use in the body.

In the case of the invasive electrochemical sensor 100, the size needs to be minimized as much as possible for reasons such as pain relief during invasion and reduction in feeling of foreign body when worn. As the size of the electrochemical sensor 400 decreases, the area of the electrodes 424 may also decrease. When the area of the electrodes 424 is not sufficiently secured, signal disturbance may occur due to noise, so it is necessary to consider both aspects of size reduction of the sensor 100 and securing of the area of the electrodes 424 during the manufacture of the electrochemical sensor 400.

The length at which the invasive electrochemical sensor 400 is inserted into the skin may be in the range of 3 to 12 mm. When the insertion length is 3 mm or less, the stability of the sensor itself and signal stability may be deteriorated due to the movement of the living body after the insertion of the sensor into the living body. When the insertion length exceeds 12 mm, the sensor is located in a range where the pain points of the human body are distributed, so that the pain becomes severe and vivo tissues such as blood vessels or nerves may be damaged. Further, the width of the invasive portion of the distal portion 406 may range from 100 to 600 μm. The thickness of the invasive portion of the distal portion 406 may be in the range of 10 to 300 μm, preferably in the range of 50 to 150 μm.

Since at least a part of the distal portion 406 is inserted into the body, when the width of the distal portion 406 is excessively wide, pain and a feeling of foreign body may increase during invasion, so it is necessary to reduce the width to a predetermined width (e.g., 600 μm) or less. When all three or more electrodes 424 are disposed only on one surface of the distal portion 406 to be inserted into the body, in terms of measurement data, the width of the distal portion 406 should be widened in order to secure a space for the three or more electrodes and the leads 426 connected thereto, but may be limited to a predetermined width (e.g., 600 μm) or less in terms of pain relief. That is, the both trade-off relationship needs to be satisfied.

The electrodes 424 of the distal portion 406 may extend along the base layer 410 through leads 426 to electrically connect to the sensor pads 428 of the proximal portion 402. Since the leads 426 is disposed in the intermediate portion 404, when the folded portion 405 is bent, the leads 426 may also be bent.

When the transmitter 200 is attached to the skin and the electrochemical sensor 400 is inserted into the body, the folded portion 405 may maintain a bent state for a considerable time. In order to reduce the torsional load of the folded portion 405, the width of the intermediate portion 404 or the folded portion 405 may be made narrower than the width of the proximal portion 402 or the distal portion 406.

The number of leads 426 provided in the intermediate portion 404 or folded portion 405 may increase in proportion to the number of electrodes disposed in the distal portion 406. As the plurality of leads 426 are disposed on the folded portion 405, insulation may deteriorate and a short-circuit may occur.

It is necessary to optimize the width between the leads 426, the number of leads 426, the number of electrodes 424, or the width of the folded portion 405.

The electrodes 424 may be disposed on both sides of the distal portion 406 to provide a sufficient space for the placement of the electrodes 424 while minimizing the size of the distal portion 406 to be inserted into the body.

By disposing the electrodes 424 of the distal portion 406 on both sides, the area of the electrodes 424 or the area of the leads 426 is sufficiently secured, so that the defect rate due to a short-circuit may be reduced, the sensitivity of the electrochemical sensor 400 may be increased, the mismatch rate may be reduced when a conductive layer 412, insulation layers 416, and the like are provided on the base layer 410, and the insertion length and width of the distal portion 406 to be inserted may be reduced.

In order to increase the accuracy in detecting electrochemical signals from the electrodes 424 of the distal portion 406, the number of working electrodes (WE) disposed on the distal portion 406 may be increased.

A plurality of working electrodes including a first working electrode and a second working electrode may be provided on the same side of the distal portion 406, or at least one working electrode may be provided on each side of the distal portion 406.

The biomarker intended to induce a reaction with the electrodes 424 may include glucose, lactose, ketone, or the like.

The first working electrode and the second working electrode may be composed of the same type of biomarker to increase the reaction force, thereby increasing the detection accuracy of the electrochemical signal. The first working electrode and the second working electrode may be composed of different type of biomarker to measure a plurality of electrochemical signals at the same time.

As such, in order to increase the accuracy of the detection of the electrochemical signal of the electrodes 424 and to keep the width of the distal portion 406 narrow, a both-surface electrode placement structure of the distal portion 406 may be required.

Referring to FIGS. 2 to 5, trenches 420 and conductive islands 430 according to the present disclosure will be described.

The electrochemical sensor 400 according to the present disclosure may include a flexible base layer 410, a conductive layer 412 provided on the base layer 410, and insulating layers 416 applied on the conductive layer 412.

The trenches 420 may be provided by laser etching the conductive layer 412. Widths W1 and W2 of the trenches 420 by laser etching may be 2 to 200 μm. The widths W1 and W2 of the trenches may be increased by moving a laser head 490 projecting a laser beam to a plurality of times to perform laser etching a plurality of times.

The electrodes 424 and the sensor pads 428 may be provided by a laser etching method by which a portion of the conductive layer is removed by projecting a laser beam to the conductive layer 412. After the conductive layer 412 is applied, an edge boundary of the electrodes 424 and an edge boundary of the sensor pads 428 may be provided. Like the electrodes 424 and the sensor pads 428, the leads 426 connecting the electrodes 424 and the sensor pads 428 may be provided by vertically cutting a portion of the conductive layer 412. After the edge boundary of the electrodes 424 and the edge boundary of the sensor pads 428 are provided, the insulating layers 416 may be attached.

The trenches 420 may be engraved into the conductive layer 412, so that conductive islands 430 may be patterned into the conductive layer 412. The height of the trenches 420 may be equal to the thickness of the conductive layer 412. The thicknesses of the conductive layer 412, the electrodes 424, and the sensor pads 428 may all be the same.

The width of the electrochemical sensor 400 may be 600 μm or less, and the length of the electrochemical sensor 400 may be 3 cm or less. The width of the electrodes 424 and the width of the sensor pads 428 may be 500 μm or less, and the width of the leads 426 may be 150 μm or less.

The electrodes 424 and the trenches 420 may be provided without burrs by laser etching even though the pattern of the electrodes 424 is complicated and the width of the trenches 420 is narrow. In order to simplify the process, the conductive layer 412 may preferably be sputtered with a metal such as gold or copper over the entire exposed area of the base layer 410. Via holes 411 extending through the base layer 410 may be provided.

Due to the via-holes 411, the conductive layers 412 applied on both sides of the base layer 410 may be electrically connected to each other.

The via-holes 411 may be provided by a laser etching method by which a part of the base layer 410 is removed by projecting a laser beam to the base layer 410.

When the both-surface electrodes 424 (i.e., the electrodes 424 provided on both surfaces) are provided, both the upper surface and the rear surface of the base layer 410 in which the via-holes 411 are provided may be sputtered with metal. The formation of the both-surface conductive layer 412 may be performed at different times on the upper surface and the rear surface, respectively, or may be performed on both surfaces at the same time.

The electrodes 424 or the leads 426 may be electrically isolated from each other by the trenches 420. As the trenches 420 are narrower, a sufficient area of the electrodes 424 for the analyte reaction may be secured. On the other hand, as the trenches 420 are narrower, insulation may deteriorate. When the trench formation is performed by laser etching, the trade-off between fine processing and insulation may be satisfied. As the width of the folded portion 405 is narrowed, the torsional force may be reduced, and fatigue failure may be prevented even when the folded portion 405 is bent and fixed for a considerable time.

Due to the trenches 420, it is easy to secure a sufficient area for the leads 426, the electrodes 424, or the sensor pads 428, thereby improving the signal transmission rate and reducing the short-circuit defect rate.

Referring to FIG. 2, the electrochemical sensor 400 may include a flexible base layer 410 that may be bent when inserted into the body. The base layer 410 may include at least one of a synthetic resin, polyimide (PI), and polyethylene terephthalate (PET) as an insulating material. Preferably, polyimide (PI) may be used as a material of the base layer 410 for obtaining a thin, flexible electrochemical sensor 400. The thickness of the base layer or the insulation layer may be 100 μm or less.

The conductive layer 412 may be provided on the base layer 410 by sputtering or the like. The thickness of the conductive layer 412 provided by sputtering metal atoms or molecules may be 10 μm or less. The conductive layer 412 may be a layer that is provided by sputtering metal over the entire exposed area of the base layer 410 before the edge boundary of the electrodes 424 and the edge boundary of the sensor pads 428 are provided.

The electrodes 424 and the sensor pads 428 may be provided by a laser etching method by which a part of the conductive layer 412 is removed by projecting a laser beam to the conductive layer 412, thereby satisfying the trade-off relationship between fine processing and insulation.

The trenches 420 may be provided in the conductive layer 412 prior to bonding the insulating layers 416 to the conductive layer 412 by means of a bonding layer 414. The conductive layer 412 may be separated into different members by the trenches 420. The conductive layer 412 may be divided into different types of electrodes 424, different leads 426, and different sensor pads 428 by the trenches 420.

After the conductive layer 412 is provided, the insulating layers 416 may be attached thereto. The insulating layers 416 may be bonded on top of the conductive layer 412 in a state in which a portion of the insulating layers 416 corresponding to the electrodes 424 and the sensor pads 428 is removed so that the electrodes 424 and the sensor pads 428 are exposed to the outside.

A portion of the insulating layers 416 may be removed by a cutter or a puncher. When the size of open areas 422 of the insulating layers 416 is small and thus micromachining is required, the laser etching method that is used to form the trenches 420 of the conductive layer 412 may be used for processing the open areas 422 of the insulation layer 412.

The base layer 410 may also be processed in the same manner. Since the via-holes 411 required to form both sides require micromachining, the laser etching method used to form the trenches 420 of the conductive layer 412 may be used to process the via-holes 411 of the base layer 410.

When the via-holes 411 are provided by cutting portions of the base layer 412, both sides of the conductive layer 412 may be sputtered with the same type of metal material seamlessly and continuously provided along the upper surface of the base layer 410, the surface of the via-holes 411, and the rear surface of the base layer 410.

The open areas 422 penetrating through the insulating layers 416 may be provided. The electrodes 424 and the sensor pads 428 provided in the conductive layer 412 may be exposed to the outside through the open areas 422. Proximal open areas 422a may be provided in the proximal portion 402 and distal open areas 422b may be provided in the distal portion 406. A portion of the sensor pads 428 may be exposed to the outside through the proximal opening 162, and a portion of the sensor pads 428 exposed by the proximal opening 162 may be electrically connected to a contact pad of the main board 202.

A portion of each of the electrodes 424 may be exposed externally through the distal opening 164, and a portion of each of the electrodes 424 exposed through the distal opening 164 may come into contact with interstitial fluid or a blood flow to electrochemically react with the analyte.

The electrochemical sensor 400 may include porous selective permeation layers 418 surrounding the surface of the electrodes 424. The selective permeation layers 418 may be applied on the electrodes 424 of the distal portion 406 for reaction with an analyte that reacts in the body.

The selective permeation layers 418 may have mesoporous characteristics. The size of the mesopores may be 2 nm to 50 nm.

The type of the selective permeation layers 418 may be determined according to the type of the analyte in the body intended to react with the electrodes 424, and may vary depending on the type of the electrodes 424 to be applied. For example, when the analyte is glucose and the electrodes 424 on which the selective permeation layers 418 are applied are working electrodes, the selective permeation layers 418 may be mesoporous platinum. Porous platinum may be fabricated from porous platinum colloids. When the analyte is glucose and the electrodes 424 on which the selective permeation layers 418 are applied are reference electrodes, the selective permeation layers 418 may be silver chloride (Ag/AgCl).

The selective permeation layers 418 may be applied on the electrodes 424 through the distal open areas 422b with the base layer 410, the conductive layer 412, and the insulating layers 416 applied. When the plurality of distal openings 422b face different types of electrodes, the first selective permeation layer 418a to the fourth selective permeation layers 418d may each include a different type of material.

The bonding layer 414 for attaching the insulating layers 416 to the conductive layer 412 may be provided. The bonding layer 414 may be disposed between the conductive layer 412 and the insulating layers 416. When the open areas 422 is provided in the insulating layers 416, the open areas 422 may also be provided in the bonding layer 414.

In order to repeatedly form the selective permeation layers 418, at least one of a dip coating method, a spray coating method, and a paste method may be performed.

The electrochemical sensors 400 may have base layers 410 connected to each other to form a sensor array. In the electrochemical sensors 400, at least one of the formation of the conductive layer 412 for each sensor on one base layer 410 and the formation of the trenches 420 by laser etching may be performed. The insulating layers 416 and the selective permeation layers 418 may also be provided at the same time.

The plurality of electrochemical sensors 400 may be individually separated from each other after the sensors are simultaneously manufactured in the form of a sensor array.

When electrodes 424 are provided on both sides of distal portion 406, the conductive layer 412 may be provided to surround the base layer 410 so that the conductive layer 412 may be provided on both sides of distal portion 406. In a state in which the open areas 422 extending through the insulating layers 416 is provided, the insulating layers 416 may be bonded to the upper side of the conductive layer 412. The electrodes 424 and the sensor pads 428 may be exposed externally through the open areas 422.

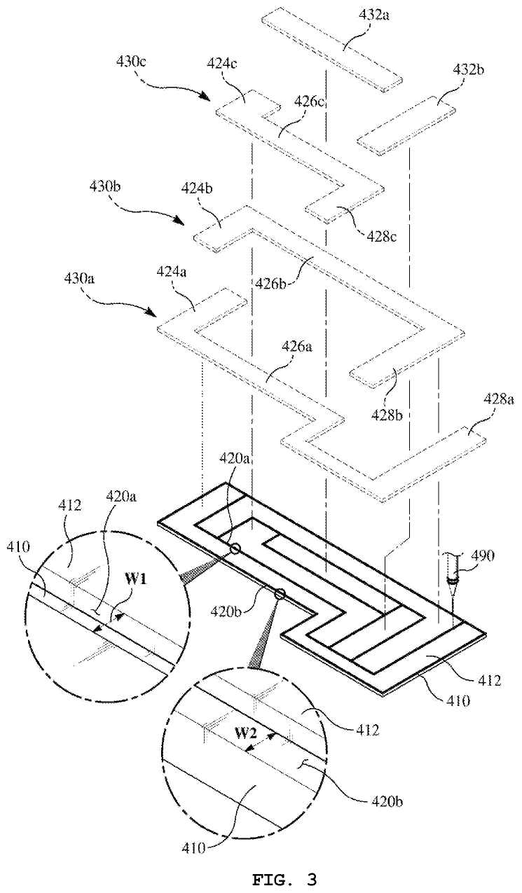

FIG. 3 schematically illustrates the entire electrochemical sensor 400 from the proximal portion 402 to the distal portion 406.

Referring to FIG. 3, after the conductive layer 412 is laminated on the base layer 410 in a manner such as sputtering, the trenches 420 may be provided by a method such as laser etching.

The conductive layer 412 may be provided with a plurality of conductive islands 430 separated from each other by laser etching or the like. Each of the conductive islands 430 may form a closed curved surface so that the conductive islands may be electrically insulated from each other.

The base layer 410 may be exposed under the trenches 420, and the adjacent conductive islands 430 may be insulated from each other by the trenches 420.

The conductive islands 430 of the proximal portion 402 may form sensor pads 428, the conductive islands 430 of the intermediate portion 404 or folded portion 405 may form the leads 426, and the conductive islands 430 of the distal portion 406 may form electrodes 424.

The conductive layer 412 may include conductive islands 430 in which portions corresponding to the electrodes 424 and the sensor pads 428 are exposed externally through the cut-out portions of the insulating layers 416, and a dummy portion 432 which is entirely covered with the insulating layers 416 so as not to be exposed externally.

The first conductive island 430a, the second conductive island 430b, and the third conductive island 430c including different electrodes 424 may be provided. The first conductive island 430a may include a first sensor pads 428a at the proximal portion 402, a first lead 426a at the folded portion 405, and a first electrode 424a at the distal portion 406.

The second conductive island 430b may include a second sensor pad 428b at the proximal portion 402, a second lead 426b at the folded portion 405, and a second electrode 424b at the distal portion 406. The third conductive island 430c may include a third sensor pad 428c at the proximal portion 402, a third lead 426c at the folded portion 405, and a third electrode 424c at the distal portion 406.

The first electrode 424a, the second electrode 424b, and the third electrode 424c may be any one of a working electrode, a counter electrode, and a reference electrode.

When the conductive islands 430 separated from each other by forming a closed curved surface are provided, the dummy portion 432 may be provided between the conductive islands 430. The dummy portions 432 may be used as conductive islands 430 having electrodes 424 or sensor pads 428 when the insulation layer is exposed. The dummy portion 432 may be completely removed by repeated laser etching or the like. However, there is no need to remove the dummy portion 432 because only electrical insulation by the trenches needs to be achieved. This is another advantage according to the present disclosure.

When the excessively wide dummy portion 432 is provided under the conductive layer 412 covered with the insulating layers 416 after the pattern of the trenches 420 is provided in the conductive layer 412, the dummy portion 432 may not be removed and remain as it is in order to prevent a portion of the insulating layers 416 from sinking downward.

The trenches 420 may include an inner trench 420a or an edge trench 420b. The inner trench 420a may insulate the conductive islands 430 from each other. The inner trench 420a may be disposed in at least one of between the electrodes 424, between the leads 426, and between the sensor pads 428.

On the other hand, when the conductive layer 412 is exposed to the edge of the electrochemical sensor 400, insulation is deteriorated, so it is necessary to prevent side exposure of the conductive layer 412. After the conductive layer 412 is applied, the conductive layer 412 may be partially cut along the edge of the electrochemical sensor 400. Thus, the edge trench 420b is provided. Accordingly, the insulating layers 416 may be attached to the edge of the electrochemical sensor 400 on the base layer 410 to provide the insulation. The insulating layers 416 may be bonded on top of the conductive layer 412 applied on the base layer 410 on the inner edge of the electrochemical sensor 400.

The edge trench 420b may form an outermost edge of conductive layer 412. The edge trench 420b may serve to insulate the conductive islands 430 positioned at the outermost portion of the electrochemical sensor 400 from the outside of the sensor 400. When the electrochemical sensors 400 are processed into an array, the edge trench 420b may prevent short-circuits between adjacent sensors 400 or separate adjacent conductive islands 430 to prevent short-circuits therebetween.

The width W1 of the inner trench 420a and the width W2 of the edge trench 420b may be in the range of 5 μm to 30 μm.

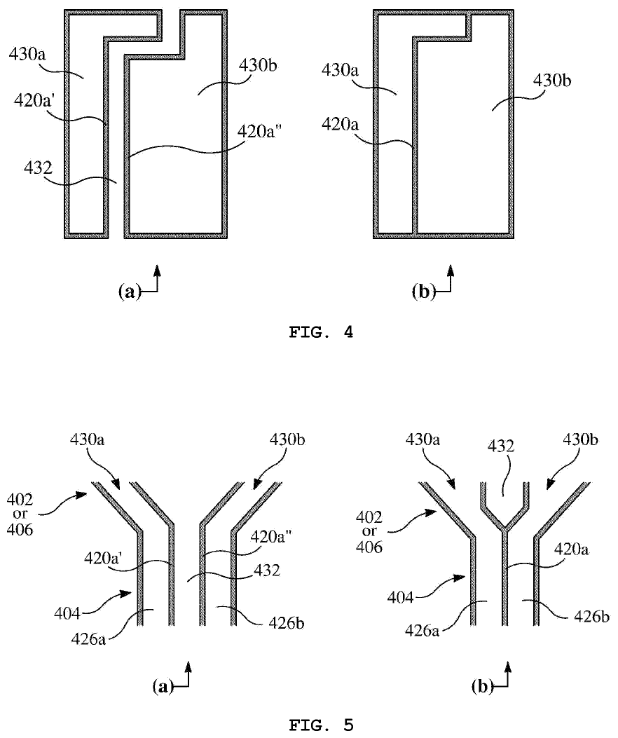

(a) of FIG. 4 illustrates an aspect in which a plurality of inner trenches 420a are provided between the conductive islands 430, and (b) of FIG. 4 may illustrate an aspect in which a single inner trench 420a is provided between the conductive islands 430.

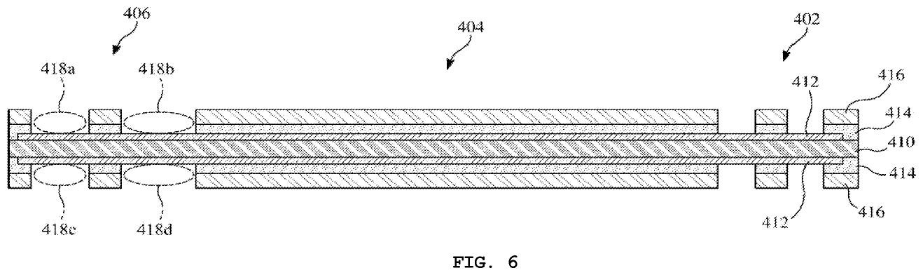

(a) of FIG. 5 illustrates an aspect in which a plurality of inner trenches 420a are provided between the leads 426 disposed in the intermediate portion 404, and (b) of FIG. 5 illustrates an aspect in which a single inner trench 420a is provided between the leads 426.

The trenches 420 may be provided in the conductive layer 412 by laser etching and removing a portion of the conductive layer 412 with a laser beam projected onto the conductive layer 412. The conductive layer 412 may be provided with the plurality of conductive islands 430 separated from each other by the trenches 420.

Due to the trenches 420, the conductive islands 430 on which the electrodes 424 and the sensor pads 428 are provided or the dummy portion 432 entirely covered with the insulating layers 416 so as not to be exposed externally may be provided on the conductive layer 412.

The dummy portion 432 may be provided between the first conductive island 430a and the second conductive island 430b, and the first inner trench 420a′ and the second inner trench 420a″ may be positioned.

Accordingly, the dummy portion 432 and at least one trench 420 may be provided between the conductive islands 430.

In this case, a short-circuit between the conductive islands 430 finely engraved by the dummy portion 432 may be prevented. Even when a factor inducing a short-circuit is generated in the electrochemical sensor 400, although the conductive island is short-circuited with the adjacent dummy portion 432, the adjacent conductive islands 430 are not short-circuited with each other, thereby reducing the probability of a measurement error.

The plurality of conductive islands 430 may share a trench 420 positioned between the plurality of conductive islands 430. In this case, the dummy portion 432 may not be provided between the first conductive island 430a and the second conductive island 430b, and one inner trench 420a may be positioned. Since the adjacent conductive islands are electrically isolated by a single shared inner trench 420a, the width of the proximal portion 402, or the width of the distal portion 406 may be reduced to minimize the size of the electrochemical sensor 400.

(a) of FIG. 5 is an enlargement of a part of (a) of FIG. 4, and (b) of FIG. 5 is an enlargement of a part of (b) of FIG. 4.

The effect of the structure of the trenches 420 illustrated in FIG. 5 may be the same as the effect of the structure of the trenches 420 in illustrated FIG. 4.

In (a) of FIG. 5, a short-circuit between the leads 426a and 426b each having a fine width may be prevented by the dummy portion 432. When the electrochemical sensor 400 has a cause for a short-circuit, a short-circuit may occur between the lead 426a or 426b and the adjacent dummy portion 432 but a short-circuit between the adjacent leads may be prevented, thereby reducing the possibility of a measurement error.

In (b) of FIG. 5, the leads 426a and 426b are electrically isolated from each other by a single common inner trench 420a, by which the width of the intermediate portion 404 may be reduced.

The sensor pads 428 may be provided on a single surface of the proximal portion 402 or both surfaces of the proximal portion 402.

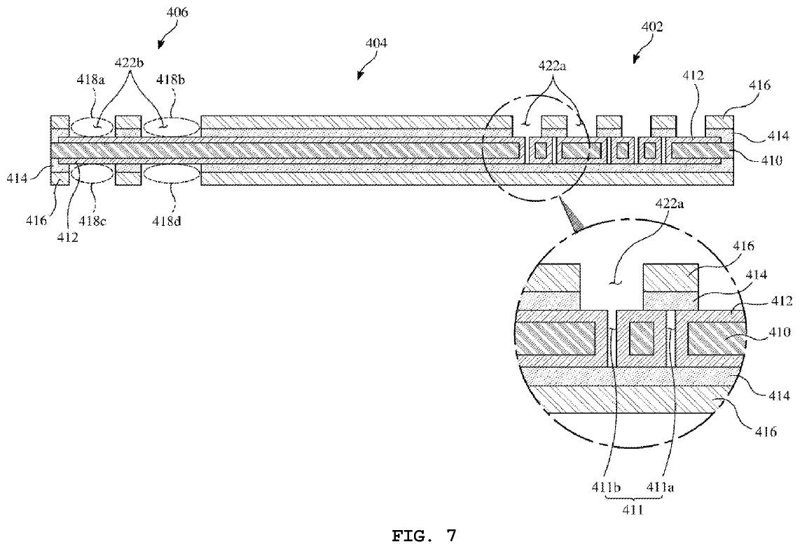

FIG. 6 illustrates a structure in which the electrodes 424 of the distal portion 406 are provided on both surfaces, in which the sensor pads 428 are provided on both surfaces of the proximal portion 402. Each of the electrodes 424 provided on both surfaces of the distal portion 406 may be electrically connected to a corresponding one of the sensor pads 428 on both surfaces of the proximal portion 402 along a corresponding one of the leads 426 provided on both surfaces of the intermediate portion 404. In this case, the via-holes 411 for electrically connecting a portion of the conductive layer 412 provided on one surface of the base layer 410 and another portion of the conductive layer 412 provided on the other surface of the base layer 410 may not be provided.

Meanwhile, when the sensor pads 428 are provided on both surfaces of the proximal portion 402, it may be difficult to electrically connect the sensor pads 428 to the contact pads of the main substrate 202 by a simple arrangement method. For example, an intercross structure or a separate connector for inserting or sandwiching the proximal portion 402 may be necessary to electrically connect the sensor pads 428 and the contact pads.

Thus, the sensor pads 428 may be provided on only one surface of the proximal portion 402.

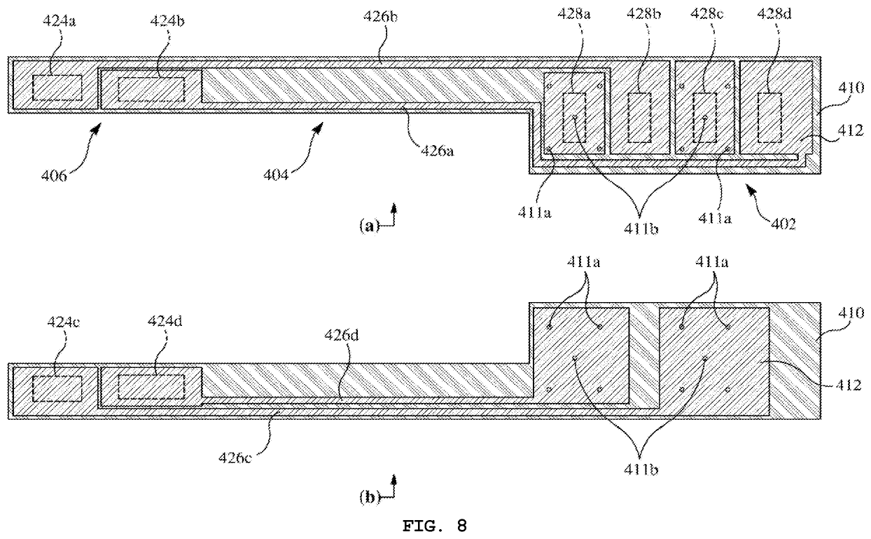

FIGS. 7 to 10 illustrate embodiments of an aspect in which the sensor pads 428 are provided on only one surface of the proximal portion 402.

The sensor pads 428 disposed on one surface of the proximal portion 402 may be arranged such that all of the sensor pads 428 are exposed in a first direction. The contact pads of the main substrate 202 may be arranged such that all of the contact pads are exposed in a second direction. The first direction and the second direction may be opposite each other by 180°. Thus, the sensor pads 428 may be electrically connected to the contact pads while facing the contact pads.

Due to the via-holes 411, the first conductive island provided on one surface of the base layer 410 and the second conductive island provided on the other surface of the base layer 410 are be electrically connected to each other. Due to the first conductive island and the second conductive island, at least one pair of components among the sensor pads 428 on one surface and the other surface, the leads 426 on one surface and the other surface, and the electrodes 424 on one surface and the other surface may be electrically connected.

The via-holes 411 may include first via-holes 411a and second via-holes 411b.

The first via-holes 411a may be blocked from the outside by the insulating layers 416.

When a portion of the distal portion 406 is inserted into a body (e.g., a human body) and an oxidation or reduction reaction occurs between the electrodes 424 and an in vivo analyte, the first via-holes 411a finely extending through the base layer 410 may be vulnerable to contamination due to reactions with a variety of body substances, since the electrochemical sensor 400 according to the present disclosure performs continuous analysis while inserted in the in vivo analyte. Thus, the first via-holes 411a may be blocked from contact with the outside by the insulating layers 416, thereby reducing contamination caused by electrochemical reactions.

At least one end of both ends of each of the second via-holes 411b may be exposed externally.

The second via-holes 411b may be provided in the electrodes 424 of the distal portion 406 or the sensor pads 428 of the proximal portion 402 that are exposed externally.

The via-holes 411 may be provided in at least one of the proximal portion 402, the intermediate portion 404, and the distal portion 406.

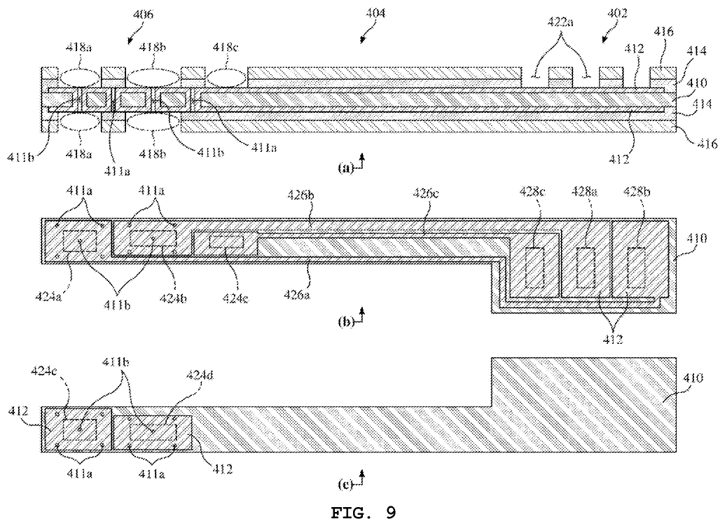

FIG. 7 and (a) and (b) of FIG. 8 illustrate a first embodiment in which the via-holes 411 are provided in the base layer 410, in which the via-holes 411 are provided in the proximal portion 402.

FIG. 7 is a side cross-sectional view illustrating a first embodiment, (a) of FIG. 8 is a plan view illustrating the first embodiment, and (b) of FIG. 8 is a bottom view illustrating the first embodiment.

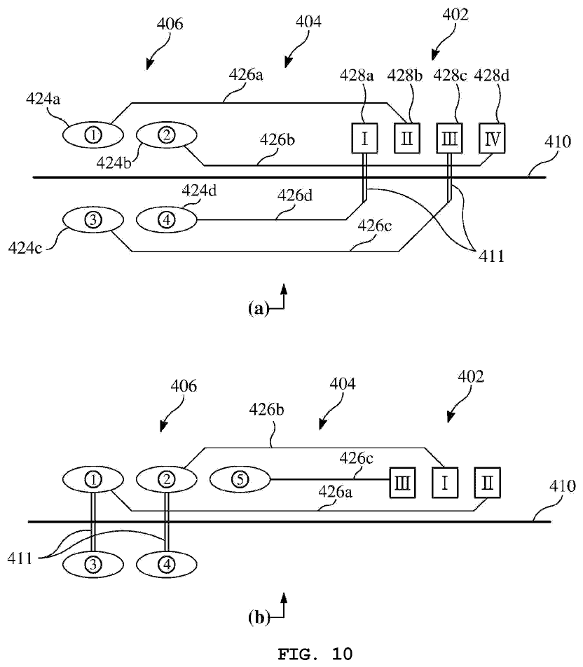

FIG. 9 illustrates a second embodiment in which the via-holes 411 are provided in the base layer 410, in which the via-holes 411 are provided in the distal portion 406.

(a) of FIG. 9 is a side cross-sectional view illustrating the first embodiment, (a) of FIG. 9 is a plan view illustrating the first embodiment, and (b) of FIG. 9 is a bottom view illustrating the first embodiment.

(a) of FIG. 10 schematically illustrates the connecting relationship of the first embodiment, and (b) of FIG. 10 schematically illustrates the connecting relationship of the second embodiment.

The first embodiment in which the via-holes 411 are provided in the proximal portion 402 and the second embodiment in which the via-holes 411 are provided in the distal portion 406 are for the convenience of description. The via-holes 411 may be provided in the proximal portion 402, the intermediate portion 404, or the distal portion 406 in an overlapping manner as required. Thus, the description of the via-holes 411 may variously be expanded according to the number and arrangement of the sensor pads 428, the number and arrangement of the electrodes 424, or the number and arrangement of the via-holes 411.

In the first embodiment, the via-holes 411 extending through the base layer 410 may be provided in the proximal portion 402.

The first electrodes exposed in the first direction may be provided on a first surface of the distal portion 406, and the second electrodes exposed in the second direction may be provided on a second surface of the distal portion 406. The first direction and the second direction opposite each other. All of the sensor pads 428 may be provided on the first surface of the proximal portion 402 to be exposed in the first direction.

Due to the via-holes 411, the second electrodes of the second surface may be electrically connected to the sensor pads 428 of the first surface in a one-to-one correspondence manner.

When the via-holes 411 are provided in the proximal portion 402, the electrodes 424 may be disposed on both surfaces of the distal portion 406. Thus, it is possible to reduce the area in which the electrodes 424 are disposed while increasing the reactivity between the in vivo analyte and the electrodes 424, thereby enabling the distal portion 406 having a smaller size to be fabricated.

Since a portion of the electrochemical sensor 400 to be inserted into the body is the distal portion 406, minimizing the size of the distal portion 406 may reduce pain and foreign body feeling of a user. The continuous analyte meter according to the present disclosure is intended for continuous measurement for a predetermined time while inserted in or attached to the body, rather than for intermittent and temporary measurement. Thus, it is important to minimize the distal portion 406.

The leads 426 connected to the electrodes 424, respectively, may also be disposed on both surfaces of the intermediate portion 404 in a distributing manner. An effect of reducing the width of the intermediate portion 404 compared to the number of the electrodes 424 provided may be obtained.

These effects of reducing the size of the distal portion 406 and reducing the width of the intermediate portion 404 may also be applied in the same manner to a situation in which the via-holes 411 are provided in the distal portion 406.

In the present disclosure, in measurement of an analyte, the transmitter 200 may be attached to the skin, the proximal portion 402 may be connected to the contact pads of the transmitter 200, and at least a portion of the distal portion 406 may be inserted into the body. When measuring the analyte in the inserted state, the electrochemical sensor 400 is required to remain in a flexibly bent state on the folded portion 405 of the intermediate portion 404. Thus, reducing the width of the intermediate portion 404 may be advantageous in terms of the reliability of measurement of the electrochemical sensor 400.

When the sensor pads 428 are disposed only on one surface of the proximal portion 402 so as to be exposed externally, the width of the proximal portion 402 may be increased. However, since the sensor pads 428 of the proximal portion 402 are electrically connected to the contact pads of the transmitter 200, there may be no problem when the width of the proximal portion 402 is greater than the width of the distal portion 406 inserted into the body.

When the via-holes 411 are provided in the proximal portion 402, the electrodes 424 provided on the distal portion 406 may be arranged in a variety of electrode patterns to have different functions. That is, the electrodes 424 disposed on the same surface of the distal portion 406 may have different functions, and the electrodes 424 disposed on both surfaces of the distal portion 406 may have different functions.

When the via-holes 411 are provided in the proximal portion 402, the insulating layers 416 may block the both one surface and the other surface of each of the first via-holes 411a from contact with the outside, thereby preventing the via-holes 411a from contamination. One end of each of the second via-holes 411b may be exposed externally through the proximal open area 422a, and the other end of each of the second via-holes 411b may be blocked from contact with the outside by the insulating layers 416.

Specifically, in the first embodiment, the first to fourth electrodes 424a to 424d may be provided, the first to fourth sensor pads 428a to 428d may be provided, and the first to fourth leads 426a to 426d connecting the electrodes and the sensor pads may be provided. The first electrode 424a and the second electrode 424b may be provided on the first surface of the base layer 410, and the third electrode 424c and the fourth electrode 424d may be provided on the second surface of the base layer 410.

The first electrode 424a may be electrically connected to the second sensor pad 428b along the first lead 426a provided on the first surface of the base layer 410. The second electrode 424b may be electrically connected to the fourth sensor pad 428d along the second lead 426b provided on the first surface of the base layer 410.

The third electrode 424c may be connected to the proximal portion 402 along the third lead 426c provided on the second surface of the base layer 410, and the fourth electrode 424d may be connected to the proximal portion 402 along the fourth lead 426d provided on the second surface of the base layer 410.

The third electrode 424c may be electrically connected to the third sensor pad 428c disposed on the first surface through a corresponding via-holes 411 in the proximal portion 402, and the fourth electrode 424d may be electrically connected to the first sensor pad 428a disposed on the first surface through a corresponding via-holes 411 in the proximal portion 402. The first lead 426a and the second lead 426b may be provided on the first surface of the base layer 410 so as not to intersect each other, and the third lead 426c and the fourth lead 426d may be provided on the second surface of the base layer 410 so as not to intersect each other.

In the first embodiment, the number of the selective permeation layers 418 provided may be the same as the number of the electrodes 424. The selective permeation layers 418 of the same type may be applied on the electrodes of the same type. In FIG. 7, when the first to fourth electrodes 424a to 424d are different types, the first to fourth selective permeation layers 418a to 418d applied on the first to fourth electrodes 424a to 424d, respectively, may be different types.

In the second embodiment, the via-holes 411 extending through the base layer 410 may be provided in the distal portion 406.

The first electrode may be provided on the first surface of the distal portion 406 to be exposed in the first direction, and the second electrode may be provided on the second surface of the distal portion 406 to be exposed in the second direction opposite the first direction.

When the via-holes 411 are provided in the distal portion 406, the first electrode and the second electrode may be electrically connected through the via-holes 411. The first electrode and the second electrode may be provided as electrodes of the same type, selected from among a working electrode, a reference electrode, and a counter electrode.

Thus, the via-holes 411 in the distal portion 406 may allow the electrodes having the same function to be disposed on both surfaces of the distal portion 406, thereby reducing measurement instability resulting from the direction in which the electrochemical sensor 400 is inserted into the body or the arrangement relationship between the needle 300 guiding the electrochemical sensor 400 inserted into the body and the distal portion 406.

When the via-holes 411 are provided in the distal portion 406, the leads 426 and the sensor pads 428 may be provided only on the first surface, and the second surface of the intermediate portion 404 or the proximal portion 402 except for the electrodes 424 of the distal portion 406 may be surrounded by the insulating layers 416, thereby being blocked from contact with the outside. Since it is not required to prepare the complicated leads 426 on both surfaces of the thin electrochemical sensor 400, the stability of flow of electricity through the entirety of the electrochemical sensor 400 including short-circuits between the leads may be improved.

When the via-holes 411 are provided in the distal portion 406, both one surface and the other surface of each of the first via-holes 411a may be blocked from contact with the outside, thereby being prevented from being contaminated. One end and the other end of each of the second via-holes 411b may be exposed externally through the distal open areas 422b.

Due to the second via-holes 411b, the electrodes of the same type may be disposed on one surface and the other surface of the base layer 410 and connected to each other. Thus, electrochemical reactivity between the electrodes 424 and the analyte may be increased irrespective of the directivity of the invasive distal portion 406.

Specifically, in the second embodiment, the first to fourth electrodes 424a to 424d may be provided, the first to fourth sensor pads 428a to 428d may be provided, and the first to fourth leads 426a to 426d connecting the electrodes and the sensor pads may be provided.

The first electrode 424a and the second electrode 424b may be provided on the first surface of the base layer 410, and the third electrode 424c and the fourth electrode 424d may be provided on the second surface of the base layer 410.

The first electrode 424a may be electrically connected to the second sensor pad 428b along the first lead 426a provided on the first surface of the base layer 410, and the second electrode 424b may be electrically connected to the first sensor pad 428a along the second lead 426b provided on the first surface of the base layer 410. A fifth electrode 424e may be electrically connected to the third sensor pad 428c along the third lead 426c provided on the first surface of the base layer 410. The present disclosure may include dummy electrodes such as the fifth electrode 424e in fabrication. The selective permeation layers 418 for reaction with the in vivo analyte may not be applied on the dummy electrodes.

The third electrode 424c disposed on the second surface of the base layer 410 may be electrically connected to the first electrode 424a disposed on the first surface of the base layer 410 through the corresponding via-holes 411 (411b), and the fourth electrode 424d disposed on the second surface of the base layer 410 may be electrically connected to the second electrode 424b disposed on the first surface of the base layer 410 through the corresponding via-holes 411 (411b). In this case, the first electrode 424a and the third electrode 424c may be electrodes of the same type, and the second electrode 424b and the fourth electrode 424d may be electrodes of the same type.

The first lead 426a and the second lead 426b may be provided on the first surface of the base layer 410 so as not to intersect each other. When the via-holes 411 are provided in the distal portion 406, the number of the leads 426 or the number of the sensor pads 428 disposed may be smaller than the number of the electrodes 424.

Unlike the first embodiment, in the second embodiment, the electrodes are connected through the corresponding via-holes 411. The selective permeation layers 418 applied on the electrodes on one surface and the other surface connected through the corresponding via-holes 411 may be the same type. For example, the first electrode 424a and the third electrode 424c may be connected through the via-holes 411b, and the same first selective permeation layer 418a may be applied on the first electrode 424a and the third electrode 424c.

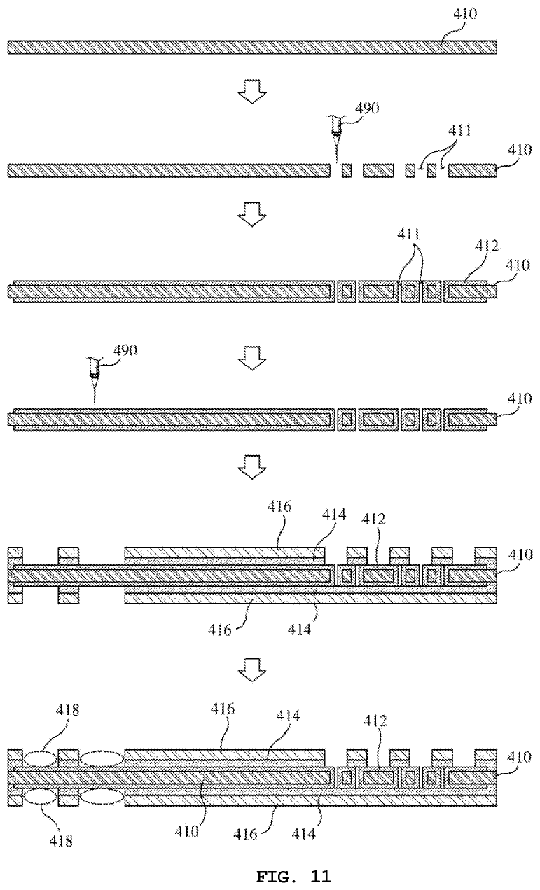

With reference to FIG. 11, a method of fabricating the electrochemical sensor 400 according to the present disclosure will be generally described.

The method of fabricating the electrochemical sensor may include: a conductive layer step of forming the conductive layer 412 on the flexible base layer 410 of the electrochemical sensor 400; and an insulating layer step of attaching the insulating layers 416 to the conductive layer 412.

The method of fabricating the electrochemical sensor may include a via-hole step of forming the via-holes 411 extending through the base layer 410.

The via-holes 411 may be formed using a laser or a mechanical method. The via-holes 411 may be formed by a laser etching method of removing portions of the base layer 410 by irradiating the base layer 410 with a laser beam generated by a laser head 490.

The via-hole step may be performed before the conductive layer step. The conductive layer step may be performed by a physical vapor deposition (PVD) method including sputtering.

The via-holes 411 may include the first via-holes 411a and the second via-holes 411b.

The first via-holes 411a may be blocked from the outside by the insulating layers 416, and at least one end of both ends of each the second via-holes 411b may be exposed externally.

The conductive layer step may include a first conductive layer step of forming a portion of the conductive layer 412 on one surface of the base layer 410 and a second conductive layer step of forming another portion of the conductive layer 412 on the other surface of the base layer 410.

Due to the first conductive layer step and the second conductive layer step, the conductive layer 412 may be formed of a single metal material continuing without a joint along the top surface of the base layer 41, the surfaces of the via-holes 411, and the bottom surface the base layer 410.

These features of the present disclosure may be different from other technologies in which a plurality of layers corresponding to the number of the electrodes 424 or the number of the sensor pads 428 are applied. When the plurality of layers corresponding to the number of the electrodes 424 or the number of the sensor pads 428 are applied on the electrochemical sensor 400 having the via-holes 411, joints such as overlaps of two or more layers may be formed around the via-holes 411. When the conductive layers are applied so that the joints such as overlaps are formed, the thickness of the conductive layer around the via-holes 411 may be uniform, thereby increasing the defective rate caused by short-circuits.

Accordingly, the conductive layer according to the present disclosure may be formed on the inner circumferential surfaces of the via-holes 411, the top surface of the base layer 410, and the bottom surface of the base layer 410 as a single layer or a substantially single layer so as to minimize short-circuit defects, such as overlaps of both ends of the via-holes 411, and reduce conduction defects of the via-holes 411.

In the insulating layer step, the first via-holes 411a may face the insulating layers 416, and the second via-holes 411b may face the open areas 422 of the insulating layers 416.

The insulating layers 416, with the open areas 422 being formed in the insulating layer step to extending through the insulating layers 416, may be bonded to the top and bottom portions of the conductive layer 412. The electrodes 424 may be exposed externally through the open areas 422 to react with the in vivo analyte. The electrodes 424 may be formed on both the top and bottom portions of the distal portion 406.

The method of fabricating the electrochemical sensor may include a trench step of forming the trenches 420 in the conductive layer 412.

The trenches 420 may be formed by laser etching of removing a portion of the conductive layer 412 using a laser beam projected to the conductive layer 412

A heat treatment step may be performed as required after the conductive layer step or the insulating layer step.

The method of fabricating the electrochemical sensor may include a selective permeation layer step of applying the selective permeation layers 418 on the open areas 422 of the insulating layers 416 by dispensing or the like.

The material of the selective permeation layers 418 may be determined depending on the type of the in vivo analyte with which the electrodes 424 are supposed to electrochemically react. For example, in the selective permeation layer step, a selective permeation layer containing platinum (Pt) may be applied on the working electrodes, and the selective permeation layer containing silver chloride may be applied on the reference electrodes.

After the selective permeation layer step, the electrochemical sensor 400 may be subjected to an additional process such as membrane coating.

With reference to FIGS. 12 to 14, the electrochemical sensor 400 according to a second aspect of the present disclosure will be described.

Referring to FIG. 12, the electrochemical sensor 400 may include: a proximal portion 10, with a plurality of sensor pads 11a, 11b, and 11c being provided on one surface of the proximal portion 10 and connected to leads 30a, 30b, and 30c, respectively; and a distal portion 20, at least a portion of which is configured to be inserted into a body (e.g., a human body).

In an embodiment of the second aspect, the electrochemical sensor 400 may include: at least one top electrode provided on a top surface of the distal portion 20 of a substrate 50; at least one bottom electrode provided on the top surface of the distal portion 20; the top leads 30a, 30b, and 30c and the bottom leads 30a, 30b, and 30c extending from the top electrode and the bottom electrode to coplanar portions of the proximal portion 10; and conductive structures 60a, 60b, and 60c comprised of at least portions of the sensor pads 11a, 11b, and 11c of the proximal portion 10 extending through the substrate 50 to be electrically connected to the bottom leads 30a, 30b, and 30c.

The electrochemical sensor 400 may refer to an electrode assembly comprised of the insulating substrate 50 and the electrodes, leads, and the sensor pads provided on the insulating substrate 50. The electrochemical sensor 400 may refer to a sensor body including a power source, a signal processor processing a signal received from the electrode assembly, a communication unit, a housing accommodating the power source, the signal processor, and the communication unit, and the like, in addition to the electrode assembly.

The sensor pads 11a, 11b, and 11c to be electrically connected to the sensor body power source, the signal processor, and the like may be provided on one surface of the proximal portion 10 of the substrate 50. A surface on which the sensor pads 11a, 11b, and 11c are provided may be referred to as a top surface for the sake of brevity.

When the electrodes and the leads 30a, 30b, and 30c are provided on both surfaces and the sensor pads 11a, 11b, and 11c are provided on both surfaces, the electrochemical sensor 400 may have a rather complicated electrical connection structure, and the size of the sensor may also be proportionally increased.

When the sensor pads 11a, 11b, and 11c are provided only one surface of the substrate 50, the electrical connection structure of the electrochemical sensor 400 may be more simplified.

The substrate 50 is not specifically limited as long as the substrate is non-toxic to the body since the substrate has to be inserted into the body and stay in the body for a long time, is strong enough to be inserted into skin tissues, is elastic enough to flexibly move along with the movement of the body, and is able to provide sufficient electrical separation between the top electrodes and the bottom electrodes.

A material of the substrate 50 meeting the above conditions may be a synthetic resin, examples of which may be, but are not limited to, polyimide (PI) or polyethylene terephthalate (PET).

In the electrochemical sensor 400 according to the present disclosure, the distal portion 20 of the substrate 50 may have a width in the range from 100 μm to 500 μm and a thickness in the range from 10 μm to 500 μm.

When the width of the distal portion 20 is smaller than 100 μm, the S/N ratio of the sensor may be reduced due to the reduced area of the electrodes, and the sensor may fail to be inserted into the body due to insufficient strength. When the width of the distal portion 20 exceeds 500 μm, problems of intensified pain and increased foreign body feeling may occur during invasion.

In the electrochemical sensor 400 according to the present disclosure, when the thickness of the distal portion 20 of the substrate is less than 10 μm, the sensor may fail to be the body due to insufficient strength. In contrast, when the thickness of the distal portion 20 of the substrate is greater than 500 μm, problems of intensified pain and increased foreign body feeling may occur during invasion.

Compared to a related-art sensor in which electrodes are provided only on one surface, the width of the electrochemical sensor 400 having the electrodes provided on both surfaces of the substrate 50 may be reduced to about half when the electrode areas (i.e., the areas of the working electrodes) are the same. Accordingly, the size of the sensor may be reduced, and user satisfaction may be improved by reducing pain caused by invasion, foreign body feeling during use, and the like.

In addition, the electrode area of the sensor having electrodes on both surfaces may be substantially doubled from the electrode area of the sensor having electrodes on a single surface when the widths are the same. The sensitivity and S/N ratio may be increased as much as the increased electrode area, thereby reducing measurement errors.

Meanwhile, the size or shape of the proximal portion 10 may be less limited than that of the distal portion 20. The proximal portion 10 may be fabricated in a suitable shape and size depending on the shape or size of the electrochemical sensor 400 or the like and the shape of the electrical connecting part.

The number or shape and the arrangement of the electrodes provided on both surfaces of the substrate 50 are not specifically limited.

For example, a single working electrode 21 may be provided on the top surface of the substrate 50, and a single counter electrode 22 may be provided on the bottom surface. Two working electrodes 21a and 21b and a reference electrode 23 may be provided on the top surface, and a single counter electrode 22 may be provided on the bottom surface. A single working electrode 21 and a single counter electrode 22 may be provided on the top surface, and a single working electrode and a single counter electrode may also be provided on the bottom surface.

These structures are illustrated in FIG. 12. Particularly, in the electrochemical sensor 400, the top electrode may be the working electrode and the bottom electrode may be the counter electrode. In contrast, the top electrode may be the counter electrode and the bottom electrode may be the working electrode.

The working electrode 21 may be formed of a porous material in order to reduce the surface area of the electrode and obtain the sensitivity of the sensor.

The glucose sensitivity of electrochemical sensor 400 using a medium porous Pt electrode may be increased to about 250 times from that of a soft Pt electrode. Glucose may be selectively detected at a higher ratio than ascorbic acid and acetaminophenol acting as interfering substances.

The electrode according to the present disclosure may be a porous Pt electrode, which may be formed of a Pt colloid.

A method of manufacturing a Pt colloid is as follows.

The method of manufacturing a Pt colloid may include: a first step of preparing a liquid composition including a surfactant and metal ions; a second step of adding a reducing agent to the liquid composition in order to form nanoparticles of a first colloid by reducing at least some of the metal ions; and a third step of removing the surfactant from the first colloid to form a second colloid that includes substantially no surfactant.

In the first step, the surfactant may have a reverse micelle phase including a number of hydrophilic spaces.

In the second step, at least some molecules of the surfactant are bonded to at least some of the nanoparticles and at least some of the plurality of hydrophilic spaces surround at least one nanoparticle, and a potential may not be applied in the process of reducing at least some of the metal ions.

In the third step, at least some of the nanoparticles may gather to form a plurality of irregularly-shaped bodies dispersed in the second colloid.

Each of the irregularly-shaped bodies may include a nanoparticle cluster including a plurality of nanoparticles.

Each of the nanoparticles has an elliptical or spherical shape with a length of about 2 nm to 5 nm. Close nanoparticles inside each cluster are spaced apart from each other to form gaps therebetween so as to be distributed across the cluster.

The colloid composition may be comprised of a first cluster and a second cluster distributed in a liquid. The length of each of the first cluster and the second cluster may range from 50 nm to 300 nm.

The first cluster may be comprised of first nanoparticles and second nanoparticles, each of which may have an elliptical or spherical shape with a diameter of about 2 nm to 5 nm.

Inside the first cluster, a first nanoparticle and a second nanoparticle are close to each other without a nanoparticle interposed therebetween while spaced apart from each other with an interparticle gap of about 0.5 nm to 3 nm therebetween.

The electrochemical sensor 400 may further include the reference electrode 23 on the top surface or the bottom surface. The reference electrode 23 may be an electrode having a constant unipolar potential. Since the unipolar potential used when measuring the electromotive force or electrode potential of a chemical cell is constant, this electrode may serve as a reference electrode. For example, the reference electrode 23 may be implemented as a silver chloride (Ag/AgCl) electrode, a calomel electrode, a mercury sulfate (I) electrode, or the like. These electrodes may also be used as the counter electrode. The silver chloride electrode may be most suitable since the electrode is used in the body.

Referring to FIG. 12, an embodiment of the second aspect may include the top leads 30a, 30b, and 30c and the bottom leads 30a, 30b, and 30c extending from the top electrodes and the bottom electrodes to coplanar portions of the proximal portion 10.

The leads 30a, 30b, and 30c serve as paths to transfer a static voltage from a power source of a main body to respective electrodes through the sensor pads 11a, 11b, and 11c and transfer current generated from electrochemical reactions generated by respective electrodes to a signal processor (not shown) of the electrochemical sensor 400 through the sensor pads 11a, 11b, and 11c.

The leads 30a, 30b, and 30c may be formed by an operation of patterning the substrate 50 with a corrosion resistant metal non-toxic to the human body, for example, gold (Au) or copper (Cu), by sputtering and then coating the patterned structure with an insulating material.

The electrochemical sensor 400 includes the conductive structures 60a, 60b, and 60c, in which portions of the sensor pads 11a, 11b, and 11c of the proximal portion 10 extend through the substrate 50 to be electrically connectable to the bottom leads 30a, 30b, and 30c.

The conductive structures 60a, 60b, and 60c may be formed by continuously applying a conductive material on at least portions of via-holes extending through the substrate 50 or filling the via-holes with the conductive material such that the sensor pads 11a, 11b, and 11c are electrically connected to the bottom leads 30a, 30b, and 30c.

The conductive structures 60a, 60b, and 60c are based on the concept of leads extending from the top to the bottom of the substrate 50 or vice versa, and electrically connect the sensor pads 11a, 11b, and 11c provided on the top surface to the leads 30a, 30b, and 30c provided on the bottom surface.

Thus, the conductive structures 60a, 60b, and 60c may be formed by disposing real leads to extend through the substrate 50 in the top-bottom direction or by forming via-holes and continuously coating portions of walls of the via-holes with a conductive material or filling the entirety of the via-holes with a conductive material.

However, it may not be easy to dispose the real leads, and thus the method of forming the via-holes and coating the via-holes with a conductive material or filling the entirety of the via-holes with a conductive material may be used.

The conductive structures 60a, 60b, and 60c may be formed by forming the via-holes, and then performing physical vapor deposition to form the leads 30a, 30b, and 30c on the top surface and the bottom surface and, at the same time, depositing the via-holes with the same material as the leads.

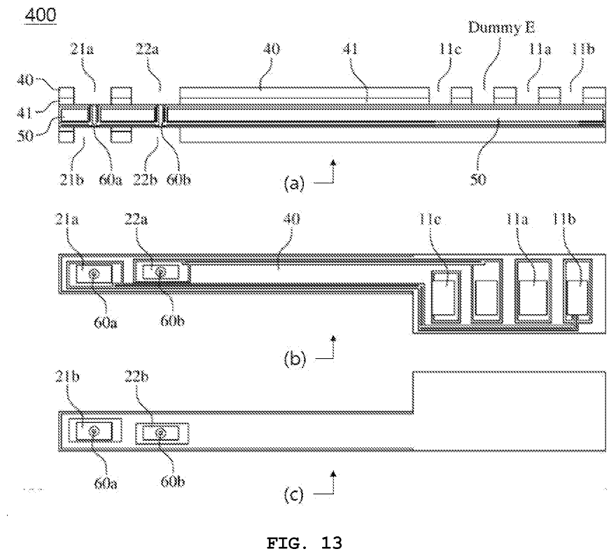

Referring to FIG. 13, another embodiment of the second aspect may include: the substrate 50 including the proximal portion 10 on which the plurality of sensor pads 11a, 11b, and 11c and the distal portion 20 configured to be inserted into a body (e.g., a human body); at least one top electrode provided on the top surface of the distal portion 20 of the substrate 50; at least one bottom electrode provided on the bottom surface of the distal portion 20 of the substrate 50; the top leads 30a, 30b, and 30c extending from the respective top electrodes to the sensor pads 11a, 11b, and 11c of the proximal portion 10; and the conductive structures 60a, 60b, and 60c extending through the distal portion 20 such that the at least one top electrode and the at least one bottom electrode are electrically connected.

The top electrodes and the bottom electrodes of the substrate 50 may be connected using the conductive structures 60a, 60b, and 60c extending through the top and bottom surfaces of the substrate 50 so as to obtain the electrode area about double the electrode area of the electrochemical sensor 400 having the electrodes on a single surface. In this case, each of the electrodes provided on the bottom surface of the substrate 50 is electrically connected to the corresponding top electrode, and thus no bottom leads are required.

The electrochemical sensor 400 according to the present disclosure may be fabricated by the following method.

First, a machining operation of, for example, cutting the substrate 50 in an intended shape and size is performed. Afterwards, the via-holes are formed in predetermined positions of the proximal portion 10 of the substrate 50 or in positions of the distal portion 20 of the substrate 50 in which the electrodes are to be formed.

The via-holes may be formed using a laser drill or a mechanical drill. The size of the via-holes may be determined in consideration of the width of the leads 30a, 30b, and 30c. The sequence of the machining operation of the substrate 50 and the operation of forming the via-holes may be reversed.

Subsequently, the leads 30a, 30b, and 30c are formed on the top surface or both surfaces of the substrate 50, physical vapor deposition (PVD), such as sputtering, is performed to continuously coat at least portions of the via-holes with a conductive material, and the shape of the leads 30a, 30b, and 30c is determined by performing laser patterning to the top surface or both surfaces.

Thereafter, insulating layers 40 having a predetermined shape are formed on both surfaces of the resultant structure, thereby determining the shapes of the electrodes, the sensor pads 11a, 11b, and 11c, and the like. Afterwards, a step of forming the electrodes by filling a frame defined by the insulating layers 40 with an electrode material or an electrode material precursor by dispensing or the like is performed. In this manner, the electrochemical sensor 400 is fabricated. In addition, after at least one of the PVD and the step of forming the electrodes, heat treatment may be performed as required.

Examples for fabricating the electrochemical sensor 400 according to the present disclosure are as follows.

### Fabrication Example 1

A GF100 25 μm product of an advanced PI material was used as a polyimide film, and 25 μm via-holes were formed in the proximal portion 10 of the polyimide film using INA SP3265 Picolaser equipment available from Ani Motion Tech Ltd. Au sputtering was performed to the top surface and the bottom surface using roll-to-roll sputtering equipment. The thickness of sputtering performed on each of the front surface and the bottom surface was about 1000 Å. Patterns of the insulating layers 40 were formed on the top surface and the bottom surface using an Asahi AZ series photoresist.

The working electrodes 21 and the reference electrode 23 were disposed on the top surface, and the counter electrode 22 was disposed on the bottom surface. Creative material 124-36 (Ag/AgCl=66:34) was dispensed on a reference electrode portion using Musashi IM-350PC, ML-5000X dispensing equipment. In addition, Pt nanoparticle ink manufactured by the applicant was dispensed on working electrode portions using Musashi IM-350PC, ML-5000X. After the dispensing was completed, machining was performed in the shape of invasive electrodes using INA SP3265 Picolaser equipment available from Ani Motion Tech Ltd. Afterwards, each of the electrodes was dipped into a Nafion 5% solution available from Merck using a dedicated dip coater.

### Fabrication Example 2

A product was fabricated in the same conditions as of Fabrication Example 1, except that a 75 μm polyimide film available from UBE was used and via-holes were formed in the proximal portion.

### Fabrication Example 3

A product was fabricated in the same manner as in Fabrication Example 1, except that eighty (80) via-holes were formed to verify the reliability of the conductivity of the via-holes.

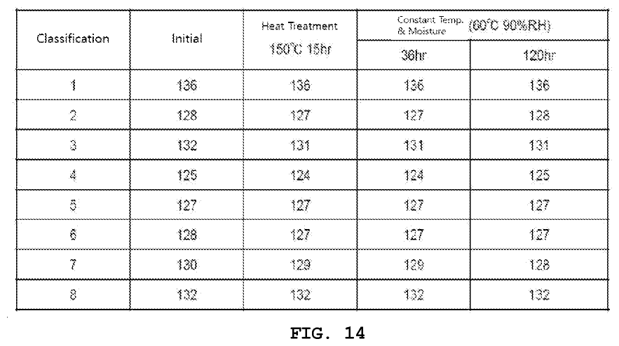

FIG. 14 is a table illustrating results obtained by measuring resistances of via-holes. The surfaces of the via-holes may be coated with the conductive material manufactured in Fabrication Example 1. Referring to FIG. 14, it is apparent that the resistance of the via-holes is significantly low and thus there are no problems in the electrical connection and reliability thereof between the sensor pads 11a, 11b, and 11c formed on the top surface and the bottom leads 30a, 30b, and 30c.

What is claimed is:

1. A continuous analyte meter comprising:
an electrochemical sensor comprising a distal portion on which a plurality of electrodes configured to react with an in vivo analyte are provided, a proximal portion on which sensor pads connected to the electrodes are provided, and an intermediate portion positioned between the distal portion and the proximal portion; and
a transmitter comprising a main substrate on which at least one of a power source, a communication unit, and a controller is provided and a housing in which the main substrate is accommodated, the transmitter being configured to be attached to the skin,
wherein the distal portion of the electrochemical sensor is provided on a portion exposed in a longitudinal direction of a needle;
the distal portion of the electrochemical sensor is configured to be inserted into a body after the skin is cut by the needle; and
the electrochemical sensor comprises a flexible base layer, a conductive layer applied on the base layer, and insulating layers attached on top of the conductive layer.

2. The continuous analyte meter of claim 1, wherein the insulating layers have open areas extending through the insulating layer and are bonded on top of the conductive layer;
the electrodes are exposed externally through the open areas; and
the electrodes are provided on both surfaces of the distal portion.

3. The continuous analyte meter of claim 1, wherein the base layer comprises via-holes extending therethrough;
the via-holes are provided by removing portions of the base layer by laser etching in which the portions of the base layer are irradiated with a laser beam.

4. The continuous analyte meter of claim 1, wherein the conductive layer comprises layer portions provided on both surfaces of the base layer by sputtering metal on the base layer.

5. The continuous analyte meter of claim 1, wherein the base layer comprises via-holes provided in cut portions thereof, and
the conductive layer comprises a single metal material continuing along and applied on a top surface of the base layer, surfaces of the via-holes, and a bottom surface of the base layer without a joint.

6. The continuous analyte meter of claim 1, wherein the sensor pads are provided only on one surface of the proximal portion;
all of the sensor pads and the contact pads are exposed in a single direction; and
the sensor pads are electrically connected to the contact pads while facing the contact pads.

7. The continuous analyte meter of claim 1, wherein the base layer comprises via-holes extending therethrough;
the conductive layer comprises layer portions applied on both surfaces of the base layer to be electrically connected to each other through the via-holes; and
the via-holes are provided on at least one of the proximal portion, the intermediate portion, and the distal portion.

8. The continuous analyte meter of claim 1, wherein the conductive layer comprises a plurality of conductive islands separated from each other, the conductive islands being separated from each other by laser etching in which portions of the conductive layer are removed with a laser beam projected on the conductive layer;
the base layer comprises via-holes extending therethrough; and
a first conductive island among the plurality of conductive islands provided on one surface of the base layer and a second conductive island among the plurality of conductive islands provided on the other surface of the base layer are electrically connected through the via-holes.

9. The continuous analyte meter of claim 1, wherein the base layer comprises via-holes extending therethrough;
the via-holes comprise a first via-hole and a second via-hole;
the first via-hole is blocked from outside by the insulating layers; and
at least one end of both ends of the second via-hole is exposed externally.

10. The continuous analyte meter of claim 1, wherein the proximal portion comprises via-holes extending therethrough;
the electrodes comprise first electrodes provided on a first surface of the distal portion to be exposed in a first direction and second electrodes provided on a second surface of the distal portion to be exposed in a second direction;
the first direction and the second direction are opposite each other;
all of the sensor pads are provided on the first surface of the proximal portion to be exposed in the first direction; and
the second electrodes on the second surface are electrically connected to the sensor pads on the first surface in a one-to-one correspondence manner through the via-holes.

11. The continuous analyte meter of claim 1, wherein the distal portion comprises via-holes extending therethrough;
the electrodes comprise first electrodes provided on a first surface of the distal portion to be exposed in a first direction and second electrodes provided on a second surface of the distal portion to be exposed in a second direction opposite the first direction;
the first electrodes and the second electrodes are electrically connected through the via-holes; and
the first electrodes and the second electrodes are electrodes of a single type selected from among a working electrode, a reference electrode, and a counter electrode.

12. The continuous analyte meter of claim 1, further comprising a plurality of leads provided on the intermediate portion to connect the electrodes and the sensor pads;
wherein the plurality of leads are provided by laser etching in which portions of the conductive layer are removed with a laser beam projected on the conductive layer; and
the leads are arranged such that each one of the leads does not cross or is twisted with another one of the leads.

13. The continuous analyte meter of claim 1, wherein the conductive layer comprises trenches therein, the trenches being provided in portions of the conductive layer removed by laser etching in which a laser beam is projected on the portions of the conductive layer;
the conductive layer comprises a plurality of conductive islands separated from each other by the trenches; and
the plurality of conductive islands share the trenches positioned between the plurality of conductive islands.

14. The continuous analyte meter of claim 1, wherein the conductive layer comprises trenches therein, the trenches being provided in portions of the conductive layer removed by laser etching in which a laser beam is projected on the portions of the conductive layer;
the conductive layer comprises conductive islands and a dummy portion divided by the trenches, wherein the electrodes and the sensor pads are provided in the conductive islands, and the dummy portion is entirely covered with the insulating layers so as not to be exposed externally; and
the dummy portion and at least one trench among the trenches are provided between the conductive islands.

15. A method of fabricating an electrochemical sensor of a continuous analyte meter for continuously measuring an in vivo analyte, the continuous analyte meter including the electrochemical sensor and a transmitter attached to the skin together with the electrochemical sensor;
wherein the electrochemical sensor includes a distal portion on which a plurality of electrodes configured to react with an in vivo analyte are provided, a proximal portion on which sensor pads connected to the electrodes are provided, and an intermediate portion positioned between the distal portion and the proximal portion, and
wherein the transmitter includes a main substrate on which at least one of a power source, a communication unit, and a controller is provided and a housing in which the main substrate is accommodated, the transmitter being configured to be attached to the skin,
the method comprising:
applying a conductive layer on a flexible base layer of the electrochemical sensor; and
attaching insulating layers to the conductive layer.

16. The method of claim 15, wherein, in the attaching of the insulating layers to the conductive layer, the insulating layers having open areas extending therethrough are bonded on top of the conductive layer;
the electrodes are exposed externally through the open areas; and
the electrodes are provided on both surfaces of the distal portion.

17. The method of claim 15, further comprising forming via-holes extending through the base layer,
wherein the via-holes are formed before the application of the conductive layer on the flexible base layer; and
the via-holes are formed by laser etching in which portions of the base layer are removed by irradiating with a laser beam.

18. The method of claim 15, further comprising forming via-holes extending through the base layer;
wherein the application of the conductive layer on the flexible base layer comprises forming a first layer portion of the conductive layer on one surface of the base layer and forming a second layer portion of the conductive layer on the other surface of the base layer; and
the conductive layer is provided as a single metal material continuing along and applied on a top surface of the base layer, surfaces of the via-holes, and a bottom surface of the base layer without a joint by the forming of the first layer portion of the conductive layer and the forming of the second layer portion of the conductive layer.

19. The method of claim 15, further comprising forming trenches in the conductive layer,
wherein the trenches are provided in portions of the conductive layer removed by laser etching in which a laser beam is projected on the portions of the conductive layer.

20. The method of claim 15, further comprising forming via-holes extending through the base layer,
wherein the via-holes comprise a first via-hole and a second via-hole;
the first via-hole is blocked from outside by the insulating layers; and
at least one end of both ends of the second via-hole is exposed externally;
in the attaching of the insulating layers to the conductive layer, the insulating layers having open areas extending through the insulating layer are bonded on top of the conductive layer; and
in the attaching of the insulating layers to the conductive layer, the first via-hole faces the insulating layers, and the second via-hole faces open areas of the insulating layers.

21. The method of claim 15, wherein, in the attaching of the insulating layers to the conductive layer, the insulating layers having open areas extending through the insulating layer are bonded on top of the conductive layer;
the method further comprising applying selective permeation layers on the open areas; and
the material of the selective permeation layers is determined depending on the type of an in vivo analyte with which the electrodes are supposed to electrochemically react.

22. An electrochemical sensor comprising:
a substrate comprising a proximal portion on which a plurality of electrodes and leads extending from the electrodes are provided, with a plurality of sensor pads connected to the leads being provided on a top surface of the substrate, and a distal portion configured to be inserted into a body;
wherein the plurality of electrodes comprise at least one top electrode provided on a top surface of the distal portion of the substrate and at least one bottom electrode provided on a bottom surface of the distal portion of the substrate; and
wherein the leads comprise top leads and bottom leads extend from the top electrodes and the bottom electrodes to coplanar portions of the proximal portion; and
conductive structures comprise some of the sensor pads of the proximal portion extending through the substrate to be electrically connected to the bottom leads.

23. The electrochemical sensor of claim 22, wherein the distal portion has a width ranging from 100 μm to 500 μm and a thickness ranging from 10 μm to 500 μm.

24. The electrochemical sensor of claim 22, wherein the top electrode is a working electrode and the bottom electrode is a counter electrode, or the top electrode is a counter electrode and the bottom electrode is a working electrode.

25. The electrochemical sensor of claim 24, wherein the electrodes further comprise a reference electrode provided on the top surface or the bottom surface of the distal portion.

26. The electrochemical sensor of claim 24, wherein the working electrode is a porous Pt electrode.

27. The electrochemical sensor of claim 26, wherein the porous Pt electrode comprises a Pt colloid.

28. The electrochemical sensor of claim 22, wherein the conductive structures comprise a conductive material continuously applied on at least portions of the via-holes extending through the substrate or the via-holes filled with the conductive material such that the sensor pads are electrically connected to the bottom leads.

29. The electrochemical sensor of claim 28, wherein the conductive material is provided by physical vapor deposition.

30. An electrochemical sensor comprising:
a substrate comprising a proximal portion on which a plurality of sensor pads are provided and a distal portion to be inserted into a body;
at least one top electrode provided on a top surface of the distal portion of the substrate and at least one bottom electrode provided on a bottom surface;
top leads extending from the top electrodes, respectively, to the sensor pads on the proximal portion; and
a conductive structure extending through the distal portion of the substrate to electrically connect the at least one top electrode and the at least one bottom electrode.

31. The electrochemical sensor of claim 30, wherein the distal portion has a width ranging from 100 μm to 500 μm and a thickness ranging from 10 μm to 500 μm.

32. The electrochemical sensor of claim 30, wherein the top electrode and the bottom electrode electrically connected through the conductive structure is a working electrode.

33. The electrochemical sensor of claim 30, wherein the electrodes further comprise a reference electrode provided on the top surface of the distal portion.

34. The electrochemical sensor of claim 30, wherein the working electrode is a porous Pt electrode.

35. The electrochemical sensor of claim 34, wherein the porous Pt electrode comprises a Pt colloid.

36. The electrochemical sensor of claim 30, wherein the conductive structure comprises a conductive material continuously applied on at least portions of the via-holes extending through the substrate or the via-holes filled with the conductive material such that the sensor pads are electrically connected to the bottom leads.

37. The electrochemical sensor of claim 36, wherein the conductive material is provided by physical vapor deposition.
